(19) United States
(12) Patent Application Publication
(10) Pub. No.: US 2026/0272527 A1
(43) Pub. Date: Sep. 17, 2026

(54) Bone Cement System And Related Assemblies And Methods

(71) Applicant: Stryker Corporation, Portage, MI (US)

(72) Inventors: Daniel Joseph Cook, Kalamazoo, MI (US); Habib Imad Baydoun, Kalamazoo, MI (US); Christopher Scott Brockman, Kalamazoo, MI (US); Gabriel James Harshman, Portage, MI (US)

(21) Appl. No.: 18/871,992

(22) PCT Filed: Jun. 7, 2023
(86) PCT No.: PCT/US2023/024680

§ 371 Date: Dec. 5, 2024

Related U.S. Application Data
(60) Provisional application No. 63/349,606, filed on Jun. 7, 2022.

(51) Int. Cl. A61B 17/88; B01F 23/50; B01F 23/53; B01F 27/1125; B01F 27/1143; B01F 27/27; B01F 101/20

(52) U.S. Cl. CPC A61B 17/8833; B01F 23/53; B01F 23/565; B01F 23/59; B01F 27/1125; B01F 27/1143; B01F 27/276; A61B 2017/8838; B01F 2101/20

(57) ABSTRACT

A bone cement system for making bone cement from bone cement components. The bone cement system includes a capital assembly (302) having a base housing. A disposable assembly (304) includes a shell (314), and a mixing chamber (318) that is enclosed within the shell by a cover (324). The disposable assembly may include a protrusion (338) removably couplable to the capital assembly. A bearing may be disposed within the protrusion. A shaft (316) driven by a rotatable drive shaft (310) of the capital assembly is coupled to the shell and is rotated by the capital assembly via inductive or mechanical power transfer. A paddle is rotatable within the mixing chamber to mix bone cement components to form bone cement with the mixing chamber enclosed within the shell. A shroud (313) may be coupled to the shell to aseptically seal a recess (311) within which a shell coupler (338) and the driven shaft is disposed.

### PRIORITY CLAIM

This application claims priority to and all the benefits of U.S. Provisional Patent Application No. 63/349,606, filed Jun. 7, 2022, the entire contents of which are hereby incorporated by reference.

### BACKGROUND

A common source of back pain is a vertebral compression fracture in which a weakened or injured vertebral body loses height or collapses. The weakening of the vertebral body may be due to acute injury or, more often, degenerative changes such as osteoporosis. One treatment modality includes vertebral augmentation in which the height of the vertebral body is elevated or restored, and stabilized at the elevated or restored height with curable bone cement. The bone cement typically includes bone cement components (e.g., a powdered polymer and a liquid monomer), which are packaged separately and mixed immediately prior to or during the vertebral augmentation procedure. Efficient, uniform, safe, and reproducible mixing of the bone cement components is an area of particular interest in development to ensure the bone cement has the expected mechanical properties and characteristics.

Known devices requiring manual mixing (e.g., “open bowl” or vacuum techniques) are inefficient by requiring operating room staff intensely agitate the bone cement components. Different staff may mix the bone cement components with varying or differing intensities and/or for varying or differing durations that may result in the bone cement not being particularly uniform or reproducible. Further, certain manual mixing devices may undesirably expose the staff to the bone cement components. Known motorized mixing devices may overcome some of the aforementioned issues, but often require disposal of a large portion of mixing components and assemblies used to form the bone cement. Disposal of these mixing components and assemblies generates excess waste and may add cost.

While mixing devices are routinely utilized to assist in the performance of a variety of different types of medical and/or surgical procedures, there is a need in the art to continuously improve such mixing devices and methods for making bone cement that overcome one or more of the aforementioned shortcomings.

### SUMMARY

A first aspect of the present disclosure provides a bone cement mixing system including a disposable assembly and a capital assembly. The disposable assembly includes a shell and a mixing chamber disposed within the shell. A shell cover removably coupled to the shell encloses the mixing chamber within the shell to provide a sterile barrier. A driven shaft is rotatably coupled to the shell and configured to receive torque from the capital assembly. A paddle is disposed within the mixing chamber and coupled to the driven shaft. The paddle is rotatable within the mixing chamber to mix bone cement components to form bone cement with the mixing chamber enclosed within the shell. The capital assembly transfers torque to the driven shaft of the disposable assembly with the shell removably coupled to the base housing.

The shell may include a blister pack. The mixing chamber may be aseptically sealed within the shell. The mixing chamber may be preloaded with the bone cement components. Alternatively, the disposable assembly may include an injection chamber coupled to the mixing chamber. The injection chamber may be preloaded with a first bone cement component and the mixing chamber may be preloaded with a second bone cement component. The first bone cement component may include a liquid monomer and the second bone cement component may include a powdered polymer.

The disposable assembly may include a shell coupler coupled to the shell and configured to releasably secure the shell to the base housing. The coupler may include a magnetic coupler or interference engagement. Alternatively, the disposable assembly may be devoid of a coupler with magnetic forces from the driven shaft and the stator being configured to maintain coupling between the disposable assembly and a base housing of the capital assembly. The shell coupler may be formed as part of the shell. The shell coupler may surround or partially surround the driven shaft.

The disposable assembly may include a delivery device disposed within the shell and removably coupled to the mixing chamber. The delivery device may define a reservoir in fluid communication with the mixing chamber. The delivery device may be configured to receive the bone cement from the mixing chamber from forces associated with the transferring of inductive power between a driven shaft disposed in the shell and a stator disposed in the capital assembly. A piston may be coupled to the driven shaft and disposed within the mixing chamber. The piston may be configured to be translated within the mixing chamber with rotation of the driven shaft to transfer the bone cement from the mixing chamber and into the reservoir of the delivery device. The delivery device may be configured to receive the bone cement from the mixing chamber with the shell cover enclosing the delivery device and the mixing chamber within the shell.

The disposable assembly may be configured to be coupled to a base housing of the capital assembly. The capital assembly may include a stator. The disposable assembly may be operably coupled with the stator of the motor such that the driven shaft is rotated relative to the shell by inductive power being transferred from the stator to the driven shaft through the shell with the shell removably coupled to the base housing. The disposable assembly may include a bearing coupled to the shell and supporting the driven shaft. The shell may include a projection defining a cavity that is contoured and sized to receive the driven shaft. The mixing chamber may be coupled to or defined by geometries within the shell. The disposable assembly further may include a gear assembly disposed within the shell and coupled between the driven shaft and the paddle to transfer torque from the driven shaft to translate the paddle.

The shell may define a recess for receiving a drive shaft of the capital assembly. The driven shaft may be disposed at least partially within the recess. The driven shaft may be aligned with a center of the recess. The mixing chamber may be disposed above the recess, or disposed adjacent the recess opposite the shell. A shroud may be removably coupled to the shell to cover the recess. The shroud may aseptically seal the recess.

The base housing may define an opening with the stator at least partially surrounding the opening. The driven shaft may be receivable within the opening such that the rotor is disposed in the opening with the shell disposed between the stator and the rotor. The driven shaft may be rotatable relative to the shell and the stator in response to inductive power being transferred through the shell from the stator. The disposable assembly may include a delivery device disposed within the shell and removably coupled to the mixing chamber. The delivery device may define a reservoir in fluid communication with the mixing chamber. The disposable assembly may include a piston coupled to the driven shaft and disposed within the shell. The piston may be translatable within the mixing chamber with the stator transferring inductive power through the shell to the rotor to rotate the rotor to transfer the bone cement from the mixing chamber and into the reservoir of the delivery device.

The stator may be configured to generate a magnetic field for transferring inductive power to the driven shaft. A sensor may be coupled to the base housing and configured to generate signals indicative of a current draw on the stator. The current draw on the stator may be based on resistance from bone cement components being mixed within the mixing chamber. A controller may be coupled to the base housing and in communication with the sensor. The controller may be configured to adjust power supplied to the stator based on the signals to facilitate consistent mixing of the bone cement components. The controller may be further configured to terminate the power supplied to the stator based on one of the signals from the sensor being a current spike indicative that a piston is prevented from further translation within the mixing chamber.

The capital assembly may include a motor coupled to the base housing to generate torque and a rotatable drive shaft extending from the base housing to transfer the torque generated from the motor to the driven shaft. The base housing may include a base coupler for coupling to the shell coupler. The recess of the shell may receive the base housing to permit coupling between the shell coupler and the motor coupler. A gear assembly may be disposed within the base housing and coupled between the motor and the rotatable drive shaft to adjust torque between the motor and the rotatable drive shaft. The capital assembly may be disposed in an aseptic enclosure.

The disposable assembly may include an injection chamber coupled to the mixing chamber. The injection chamber may be preloaded with a first bone cement component and the mixing chamber being preloaded with a second bone cement component. The disposable assembly may include an actuator to move the first bone cement component from the injection chamber to the mixing chamber. A sensor may be coupled to the base housing and configured to generate signals indicative of a coupling between the shell coupler and the base coupler. A controller may be coupled to the base housing and in communication with the sensor and the actuator. The controller may be configured to operate the actuator to move the first bone cement component from the injection chamber into the mixing chamber based on the signal from the sensor indicating the shell coupler is coupled to the base coupler. At least one of the shell coupler and the base coupler may include a magnetic coupler or interference engagement.

A second aspect of the present disclosure provides a method of forming bone cement with a bone mixing system including a capital assembly having base housing, a stator, and a controller. The method includes initiating, with the controller, power being supplied from a power source to the stator. The method also includes transferring inductive power from the stator through the shell to the rotor. The method further includes mixing, with the paddle, the bone cement components in the mixing chamber to form bone cement. The method also includes translating the piston with the mixing chamber to compress the bone cement. The method further includes detecting, with a sensor, a current spike indicative that the piston is prevented from further translation within the mixing chamber. The method also includes terminating, with the controller, the power supplied to the stator based on a signal from the sensor. The step of mixing the preloaded bone cement components may be performed while an interior of the shell is aseptically sealed.

A third aspect of the present disclosure provides a method of forming bone cement with a bone mixing system including a capital assembly having a base housing, a motor, and a rotatable drive shaft, and a disposable assembly including a shell within which a mixing chamber, a paddle, a delivery device, and bone cement components are sealed with a shell cover. The method includes coupling the shell to the base housing. The method also includes providing an input to operate the motor to rotate the paddle within the mixing chamber to mix the bone cement components to form bone cement and transfer the bone cement to the delivery device while the shell is sealed. The method further includes unsealing the shell to access the delivery device and removing the delivery device from the shell. The method may include introducing a first bone cement component from outside the mixing chamber to a second bone cement component disposed inside the mixing chamber after coupling the shell to the base housing. The step of transferring the shell into a sterile field of an operating suite may occur before the step of unsealing the shell to access the delivery device. The step of unsealing the shell to access the delivery device may occur after the bone cement is mixed and transferred to the delivery device.

A fourth aspect of the present disclosure provides another bone cement system. The bone cement system may include a driver having a motor and a driver coupler configured to transfer torque. The bone cement system may also include a disposable assembly having a mixing chamber. A paddle may be disposed within the mixing chamber. A mixing coupler may be coupled to the paddle and configured to be coupled to and receive torque from the driver coupler to rotate and translate the paddle within the mixing chamber. A delivery device may be removably coupled to the mixing chamber. The delivery device may have a delivery coupler configured to couple to and receive torque from the driver coupler for introducing bone cement received from the mixing chamber to a surgical site. The driver may be a handheld driver. The driver may be disposed within an aseptic enclosure. A bone cement component injector may be coupled to the mixing chamber to introduce a bone cement component to the mixing chamber. The bone cement component injector may include an actuator operable to move the bone cement component into the mixing chamber. The bone cement injector may be detachable from the mixing chamber.

A fifth aspect of the present disclosure provides a method of forming bone cement with a bone mixing system including a driver and a disposable assembly including a mixing chamber having a paddle, and a delivery device. The method includes coupling a driver coupler of the driver to a mixing coupler of the mixing chamber. The method also includes operating the driver to rotate the paddle within the mixing chamber to mix bone cement components to form bone cement and transfer the bone cement to the delivery device. The method further includes decoupling the driver coupler from the mixing coupler. The method also includes coupling the driver coupler to a delivery coupler of the delivery device. The method further includes operating the driver to introduce mixed bone cement to a surgical site. The disposable assembly may further include a bone cement injector. The method may further include introducing a first bone cement component with the bone cement injector from outside the mixing chamber to a second bone cement component disposed inside the mixing chamber. The method may also include detaching the mixing chamber from the delivery device after the step of operating the driver to transfer bone cement to the delivery device.

A sixth aspect of the present disclosure provides another bone cement system having a power pad and a bone cement mixing assembly. The power pad has a power transmitter. The bone cement mixing assembly may include a mixing chamber and a paddle rotatable within the mixing chamber to mix bone cement components into bone cement. A motor may be operably coupled to the paddle to rotate the paddle. A controller may be coupled to the motor and configured to operate the motor to generate torque and rotate the paddle. A receiver may be coupled to the controller and the motor. The transmitter and the receiver may collectively form a transformer for inductive power coupling between the power pad and the bone cement mixing assembly. The controller may be configured to operate the motor to rotate the paddle responsive to the transmitter and the receiver being inductively coupled. The transmitter and the receiver may each include a coil.

### BRIEF DESCRIPTION OF THE DRAWINGS

FIG. 1 is a perspective view of a first implementation of a bone cement system in which a capital assembly and a disposable assembly are configured to inductively engage one another.

FIG. 2 is a schematic plan view of a first variant of the disposable assembly.

FIG. 3 is a schematic plan view of a rotor of the disposable assembly coupled to the capital assembly.

FIG. 4 is a schematic plan view of the rotor of the disposable assembly decoupled from the capital assembly.

FIG. 5 is a schematic plan view of a second variant of the disposable assembly.

FIG. 6 is a bottom perspective view of a second implementation of the disposable assembly. A shroud is shown as decoupled from a recess of the disposable assembly to expose a shell coupler.

FIG. 7 is a perspective view of the step of the disposable assembly being coupled with the capital assembly.

FIG. 8 is a perspective view of the bone cement system being activated with the disposable assembly coupled to the capital assembly.

FIG. 9 is a perspective view of the step of the disposable assembly being decoupled from the capital assembly.

FIG. 10 is a perspective view of the step of a cover being removed from a shell of the disposable assembly.

FIG. 11 is a perspective view of the step of a delivery device being removed from the shell of the disposable assembly.

FIG. 12 is a plan view of another bone cement system in which a driver is configured to removably engage a third implementation of the disposable assembly.

FIG. 13 is an elevation view of the bone cement system of FIG. 12 with the driver coupled to a mixing coupler and an injection actuator in a first configuration.

FIG. 14 is an elevation view of the bone cement system of FIG. 12 with the driver coupled to the mixing coupler and the injection actuator in a second configuration.

FIG. 15 is an elevation view of the driver coupled to a delivery device from the bone cement system of FIG. 12.

FIG. 16 is a perspective view of another bone cement system in which a power paid is configured to wirelessly transfer power to the disposable assembly.

### DETAILED DESCRIPTION

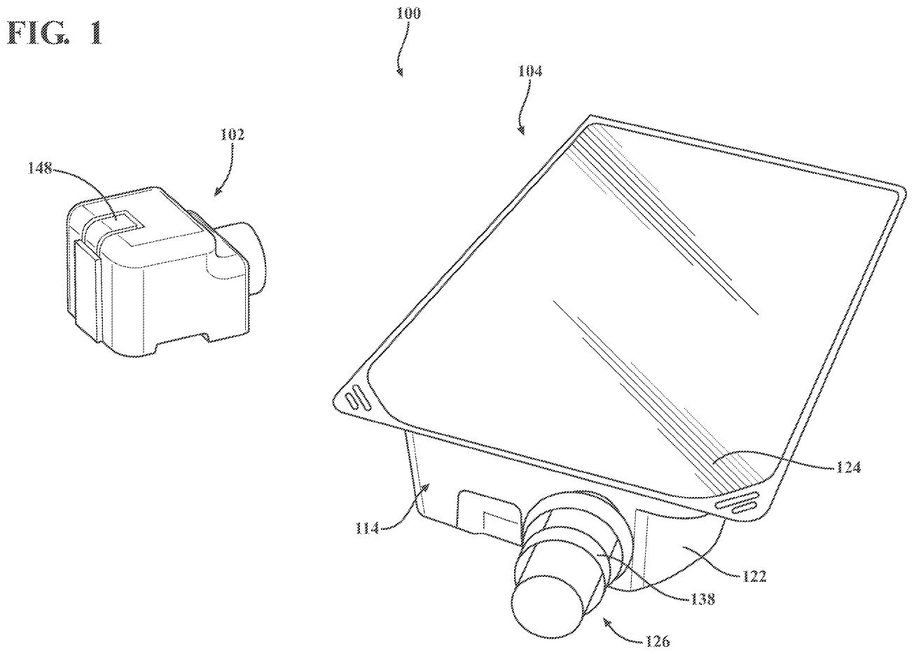

Referring now to the figures, wherein like numerals indicate corresponding parts throughout the several views, a bone cement system 100 is shown in FIG. 1. The system 100 includes a capital assembly 102, and a disposable assembly 104 configured to be removably coupled to the capital assembly 102. Through a sterile barrier, the capital assembly 102 operates components within the disposable assembly 104 to mix bone cement components to form bone cement. Among other advantages to be described, the inductive or mechanical power being transferred through the sterile barrier limits user exposure to the bone cement components. A motor and/or electronics may be provided on the capital assembly 102, thereby rendering the disposable assembly 104 less expensive to manufacture. Furthermore, the advantageous arrangement provides for the capital assembly 102 to be reused in subsequent surgical procedures with a different disposable assembly 104, thereby providing a more environmentally sustainable system. The system 100 may be useful for any procedure in which the delivery of bone cement to a target site is indicated, for example, vertebroplasty, kyphoplasty, bone tumor ablation, and the like.

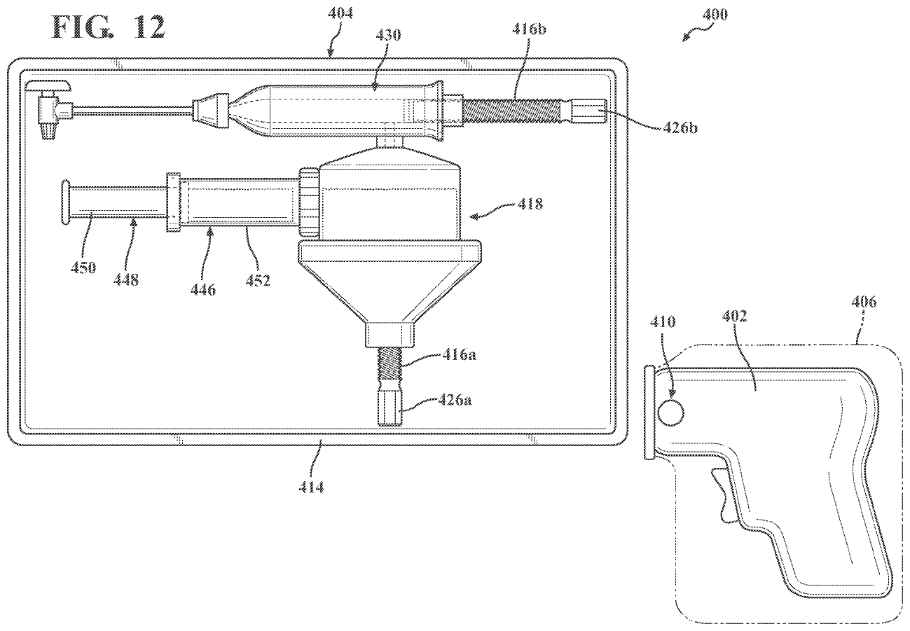

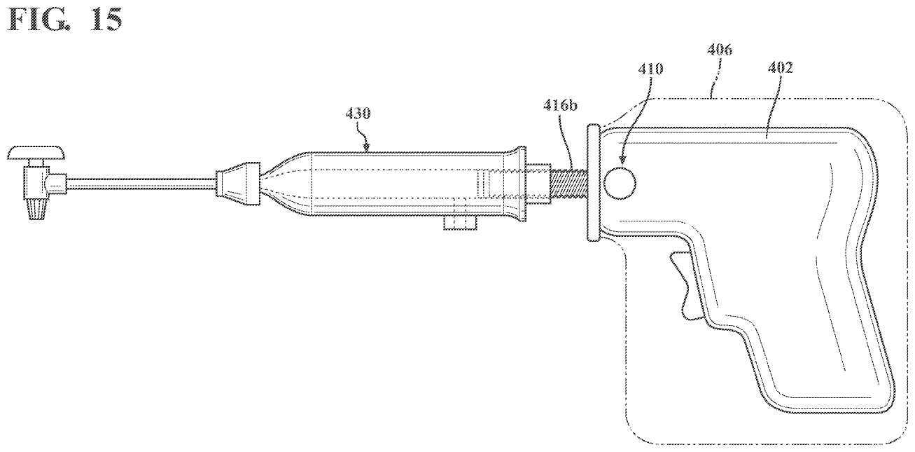

A first implementation of the bone cement system 100 is shown in FIGS. 1-5 in which inductive power is transferred through the sterile barrier provided by the disposable assembly 104. The capital assembly 102 includes a base housing 106 defining an opening 108 for at least partially receiving a portion of the disposable assembly 104. Alternatively, the arrangement may be reversed in which the disposable assembly 104 defines an opening for at least partially receiving a portion of the base housing 106. The base housing 106 may be dimensioned to be freestanding on a support surface, such as a surgical stand or Mayo stand/table. The opening 108 may be defined with a side of the base housing 106 at a height complementary to that of a projection 126 of the disposable assembly 104 to be situated next to the base housing 106 on the support surface. Alternatively, the capital assembly 102 may be handheld and configured to be manually supported and actuated by the user (see, e.g., FIGS. 12-15).

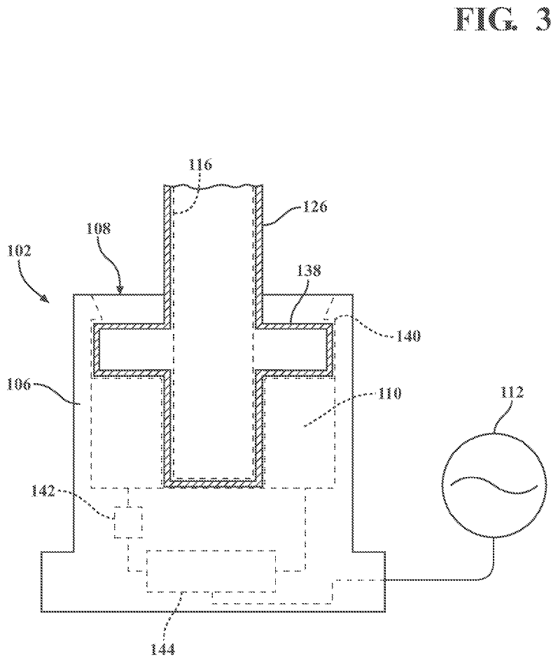

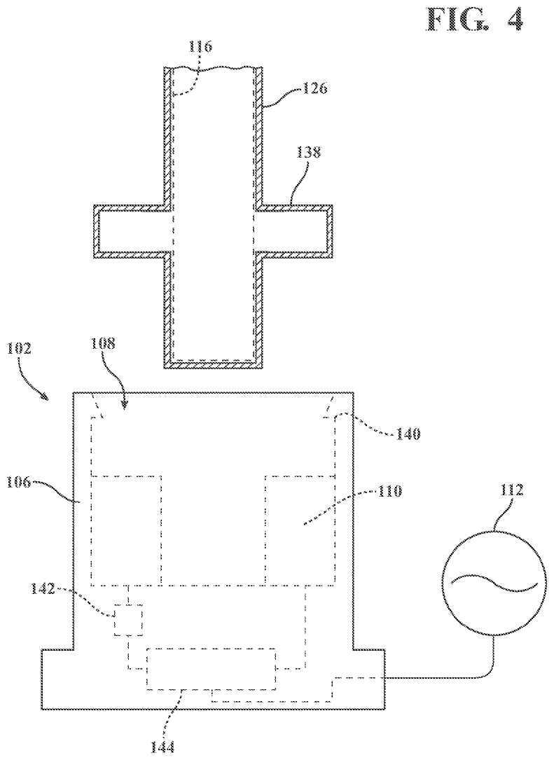

As best shown in FIGS. 3 and 4, the capital assembly 102 includes a stator 110 coupled to the base housing 106. The stator 110 may include a coil surrounding the opening 108 of the base housing 106. A power source 112 may be disposed on the base housing 106 (e.g., a battery) to power the stator 110 and generate a magnetic field within the opening 108, or alternatively, the capital assembly 102 may be configured to be coupled to the power source 112 (e.g., a wall-mounted outlet). The capital assembly 102 may include a sensor 142 configured to generate signals indicative of a current draw on the stator 110. The current draw on the stator 110 may be based on resistance from the bone cement components being mixed within a mixing chamber 118. The capital assembly 102 may also include a controller 144 coupled to the base housing 106 and in communication with the sensor 142. The controller 144 may be configured to adjust power supplied to the stator 110 based on the signals from the sensor 142 to facilitate consistent mixing of the bone cement components. For instance, changes in viscosity of the bone cement mixture in the mixing chamber 118 may result in in current draw on the stator 110, and detected by the sensor 142. The sensor 142 may be configured to generate signals indicative of a current viscosity of the bone cement mixture, and the controller 144 may be configured to increase or decrease power to the stator 110 if the signal indicates the bone cement does not have the desired viscosity. Further, the controller 144 may be configured to terminate power being supplied to the stator 110 if the signal indicates the bone cement has the desired viscosity, or at completion of the mixing and transferring phases of the operational cycle.

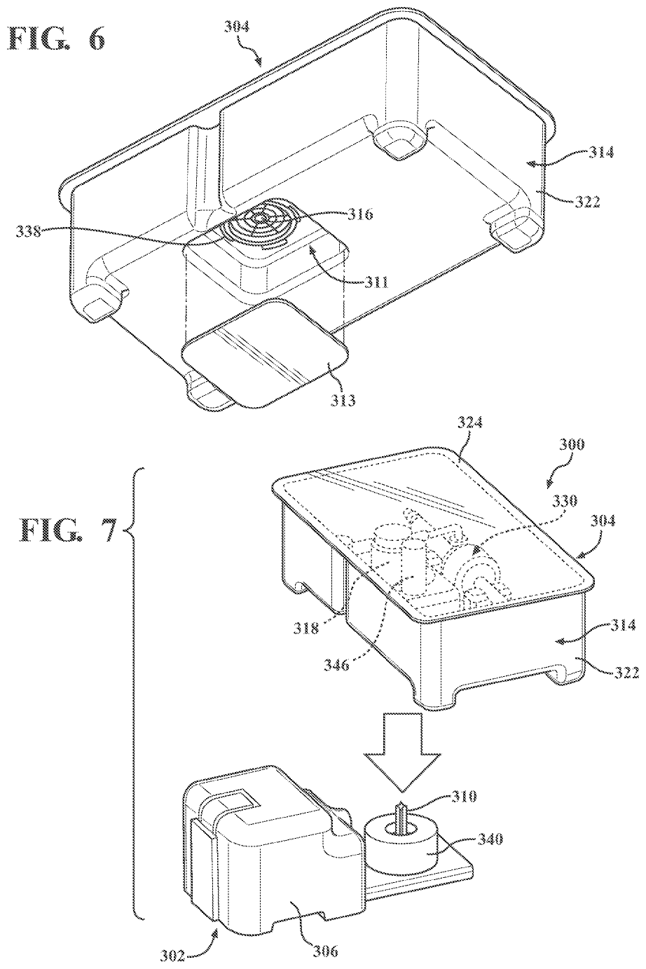

The disposable assembly 104 includes a shell 114, and a rotor 116 rotatable relative to the shell 114. The rotor 116 is disposed within the shell 114, and more particularly it may be fully encapsulated within the shell 114 so as to be sterile. In other words, the shell 114 in combination with at least one cover 124-provides a sterile barrier. The shell 114 may include a projection 126, and the rotor 116 may be rotatably disposed within the projection 126. The projection 126 and the rotor 116 are configured to at least partially be disposed within the opening 108 of the base housing 106 to facilitate magnetic communication between the stator 110 and the rotor 116. In response to inductive power (resulting from generation of the magnetic field) being transferred from the stator 110 to the rotor 116, the capital assembly 102 operates components of the disposable assembly 104 without compromising the sterile barrier. As best shown in FIGS. 3 and 4, the projection 126 defines a cavity that is contoured and sized to receive the rotor 116 and to permit rotation of the rotor 116, but otherwise restrict movement of the rotor 116. The projection 126, in the illustrated implementation, extends out of a sidewall of the shell base 122; however, other locations for the projection 126 are contemplated based on the dimensions of the shell 114. In one non-limiting example, the shell 114 may define a recess (see, e.g., FIG. 6) within which the projection 126 is disposed.

A bearing 128 may be disposed in the shell 114. In the illustrated implementation, the shell 114 may be contoured around the bearing 128 to constrain the position of the bearing 128 within the shell 114. In other configurations, the shell 114 may include a cavity for receiving the bearing 128 and the bearing 128 may be press fit in the cavity, or supported by another subcomponent. The bearing 128 may include a ball bearing, a journal bearing, or another suitable friction reducing mechanism that may support the rotor 116 and prevent wear of the rotor 116 against the shell 114 and/or wear of the shell 114 from the rotor 116. It is understood that the bearing 128 is optional.

As mentioned, the disposable assembly 104 is removably coupled to the capital assembly 102. Referring again to FIGS. 3 and 4, the base housing 106 may include a base coupler 140, and the shell 114 may include a shell coupler 138 to facilitate the removable coupling between the disposable assembly 104 to the capital assembly 102. The couplers 138, 140 may be, for example, interference couplers where at least one of the base and shell couplers 138, 140 deflect during insertion of the projection 126 of the shell 114 of the disposable assembly 104 in the opening 108 of the base housing 106. The retention force is sufficient to retain the engagement until greater force is applied by the user to remove the disposable assembly 104 from the capital assembly 102. The shell coupler 138 may be formed from resiliently deflectable material of at least a portion of a shell base 122 and/or the projection 126. The shell coupler 138 may be disposed further outwardly from the sidewall of the shell base 122 than the bearing 128. Additionally or alternatively, the base and shell couplers 138, 140 may be magnetic couplers that possess sufficient magnetic force to retain the disposable assembly 104 to the capital assembly 102, but permit a user to separate when desired. In still other configurations, the disposable assembly 104 and the capital assembly 102 are devoid of couplers 140, 138. Instead, the magnetic forces from the rotor 116 being disposed in the magnetic field of the stator 110 may be configured to maintain coupling between the disposable assembly 104 and the base housing 106 of the capital assembly 102. More specifically, because the position of the rotor 116 is constrained within the shell 114 of the disposable assembly 104 (except for rotation of the rotor), magnetic forces between the rotor 116 and the stator 110 may maintain engagement between the shell 114 to the base housing 106. It is further contemplated that resistance from compression of the bone cement within the mixing chamber 118 may also facilitate maintaining operable engagement between the rotor 116 and the stator 110.

The shell 114 may include a blister pack having the shell base 122, and a cover 124 removably coupled to the shell base 122. The shell base 122 may be a thermoformed plastic generally contoured to house the components of the disposable assembly 104. The cover 124 may include a peel-away film coupled to the shell base 122 with adhesive. The shell base 122 and the cover 124 may collectively form the sterile barrier. In other words, the contents of the disposable assembly 104 and the capital assembly 102 are aseptically sealed.

A mixing chamber 118 is disposed within the shell 114. The mixing chamber 118 may be defined by geometries formed within the shell 114, such as through a thermoforming operation. Alternatively, the mixing chamber 118 may be a separate subcomponent or substructure supported within the shell 114 with the geometries of the shell 114 preventing or limiting movement of the mixing chamber 118. An adhesive or other joining means may also fix in place the mixing chamber 118 within the shell 114.

The delivery device 130 may be supported within the shell 114 and define a reservoir 132 in fluid communication with the mixing chamber 118. The reservoir 132 of the delivery device 130 is configured to receive the bone cement from the mixing chamber 118. The delivery device 130 is separable from the disposable assembly 104. In other words, bone cement is mixed and transferred to the delivery device 130, after which the cover 124 is unsealed from the shell base 122 for the delivery device 130 to be removed and used. Thus, the user need not access the sealed components of the system 100 until the bone cement is already mixed and transferred to the delivery device 130. Examples of the delivery device 130 suitable for the present system is disclosed in commonly-owned International Patent Publication No. WO 2019/200091, published Oct. 17, 2019, and commonly-owned International Patent Publication No. WO 2020/252296, published Dec. 17, 2020, the entire contents of each being hereby incorporated by reference. Another example of the delivery device 130 suitable for the present system is disclosed in commonly owned U.S. Pat. No. 6,547,432 , issued Apr. 15, 2003, the entire contents of which is hereby incorporated by reference. In an alternative implementation, the mixing chamber 118 and the delivery device 130 may be functionally integrated. One example includes the mixing chamber 118 being separable from the shell 114 and the rotor 116 configured to dispense the bone cement directly from the mixing chamber 118. A handle or other actuator may be operably coupled to the mixing chamber 118 to do so.

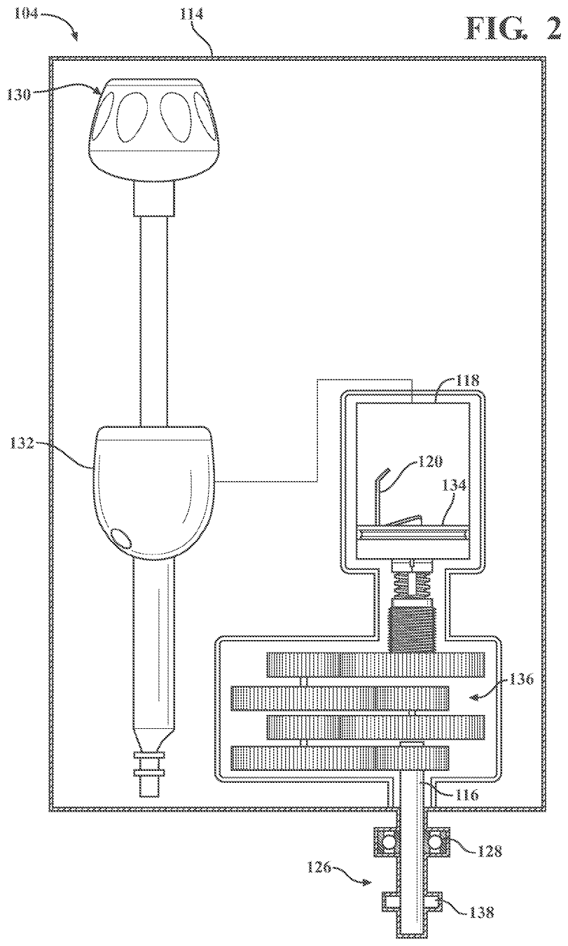

In a first variant and with reference to FIG. 2, a paddle 120 is coupled to the rotor 116 and disposed within the mixing chamber 118. The paddle 120 is configured to rotate in response to rotation of the rotor 116. The paddle 120 is configured to mix the bone cement components to form bone cement. The paddle 120 may be configured to buckle against a distal wall of the mixing chamber 118 with translation of a piston 134 to which the paddle 120 is coupled. The disposable assembly 104 may further include a gear assembly 136 (e.g., a gear train) disposed within the shell 114 coupled to the rotor 116 and configured to translate or advance the piston 134 within the mixing chamber 118 while also rotating the paddle 120. The paddle 120 within the mixing chamber 118 mixes bone cement components to make the bone cement, and the piston 134 compresses and transfers the bone cement to the delivery device 130. Thus, the delivery device 130 becomes ready to use without the sterile barrier being violated. An example of such a configuration of the mixing chamber 118, the paddle 120, the piston 134, and the gear assembly 136 suitable for the present system is disclosed the aformentioned International Patent Publication No. WO 2020/252296. In other configurations, the rotor 116 may be coupled directly to the piston 134 to advance the piston 134 and rotate the paddle 120 within the mixing chamber.

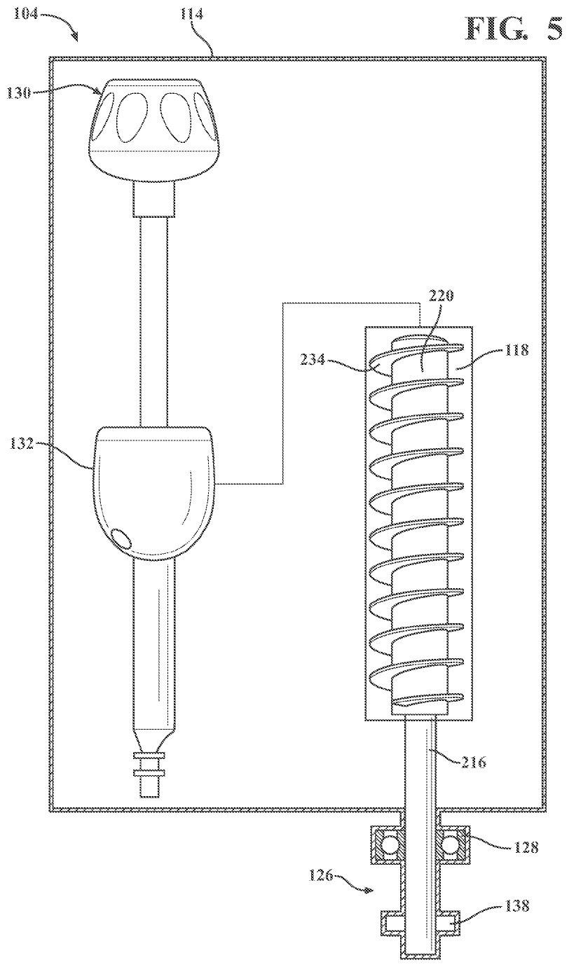

In another variant of the disposable assembly 104 shown in FIG. 5, with like numerals indicating like components. An auger 220 may be disposed within the mixing chamber 118. Helical screw blades 234 of the auger 220 may be used to facilitate mixing of the bone cement components. The auger 220 may be rotated in response to rotation of the rotor 116 to mix the bone cement components and advance the bone cement to the reservoir 232 of the delivery device 230. The auger 220 may be coupled directly to the rotor 216. In other configurations, a gear assembly 236 may be disposed between the rotor 216 and the auger 220.

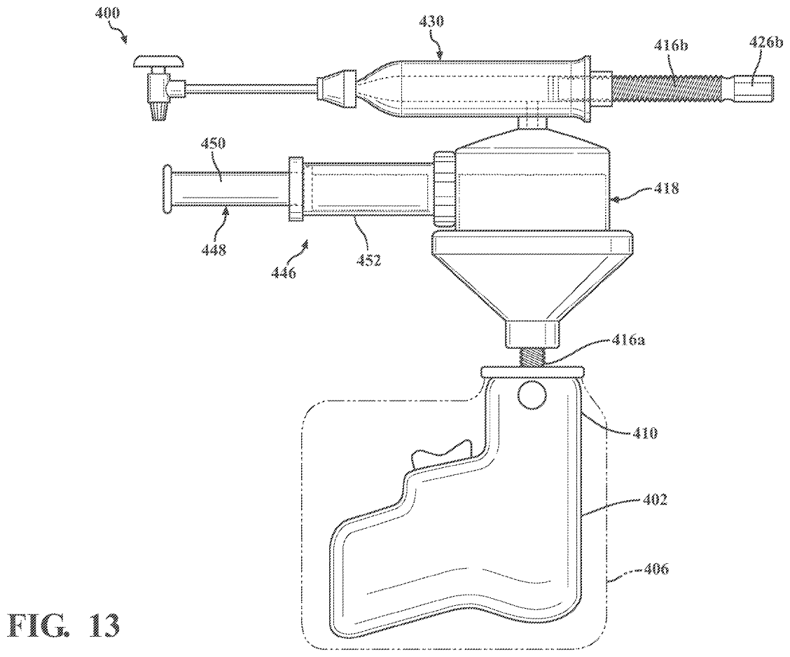

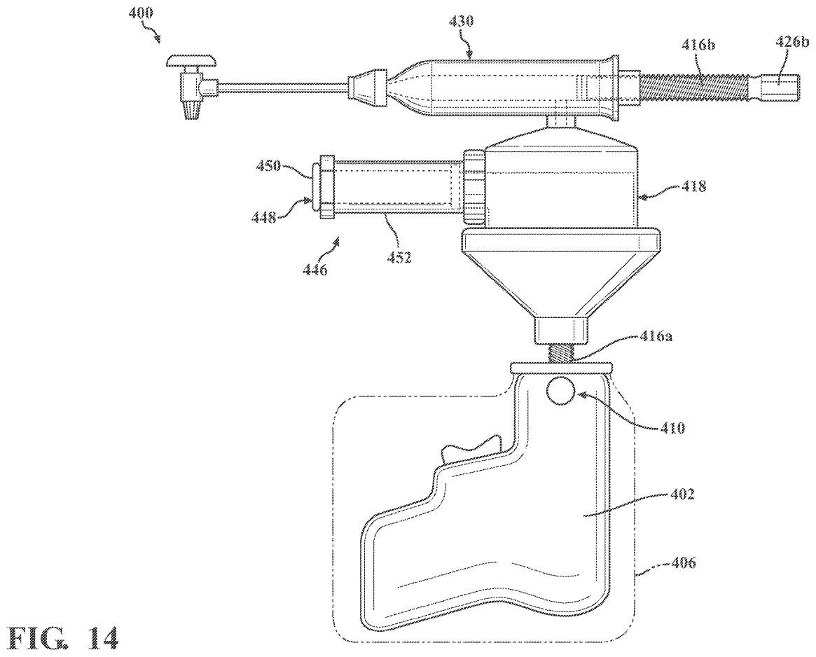

As mentioned, the bone cement components are mixed within the mixing chamber 118. Typically, the bone cement components include a powdered polymer and a liquid monomer. The powdered polymer may be preloaded into the mixing chamber 118. Prior to mixing, the liquid monomer is loaded into the mixing chamber 118 either automatically or manually, in either instance with the cover 124 still sealed to the shell base 122. In one example, one of the bone cement components may be disposed in a separate chamber in selective communication with the mixing chamber 118 so that mixing of bone cement components does not occur until mixing is desired by a user. The other of the bone cement components (e.g., the liquid monomer) may be disposed within an ampule (not shown) within the shell 114, and operation of the rotor 116 from the stator 110 may cause the ampule to break or “crack” to introduce the monomer with the powdered polymer that was preloaded in the mixing chamber 118. In another example, a monomer injection mechanism (see, e.g., FIGS. 13 and 14), such as a small, syringe-like mechanism, may be integrated with the shell base 122 and preloaded with the liquid monomer. The monomer injection mechanism is configured to receive an input from the user (e.g., twist input, push input, etc.) to cause the preloaded liquid monomer to be injected and directed into the mixing chamber 118. In still another example, the shell base 122 may define a port (not shown), such as a Luer fitting, configured to be removably engage the monomer injection mechanism packaged separately from the disposable assembly 104. The Luer fitting may be configured to accept the liquid monomer from a syringe, a pouch, an ampoule cracking device, or via direct pour of the liquid monomer from the ampoule. It is contemplated that the bone cement components may be introduced using other methods without requiring the sterile barrier being violated.

Therefore, exemplary methods of operation of the bone cement system 100 facilitate improved sterile handling within the surgical suite. The capital assembly 102 may be supported on the surgical stand, as mentioned, and positioned within a non-sterile area of the surgical suite. The user retrieves the disposable assembly 104, and situates it on the surgical stand next to the capital assembly 102. If applicable, the user operates the monomer injection mechanism or otherwise causes the liquid monomer to mix with the powered polymer that may be preloaded in the mixing chamber 118.

The user couples the disposable assembly 104 to the capital assembly 102. For example, the shell 114 of the disposable assembly 104 may be removably coupled to the base housing 106 of the capital assembly 102 such that the rotor 116 is in magnetic communication with the stator 110. The user may actuate an actuator 148 to initiate the operational cycle, or alternatively the controller 144 may be configured to provide power to the stator 110 automatically upon detection of the magnetic communication. In response to power being supplied to the stator 110, inductive power is transferred from the stator 110 to rotate the rotor 116 within the mixing chamber 118 to facilitate mixing the bone cement components, and transfer the bone cement to the delivery device 130. The sensor 142 may generate the signals responsive to changes in current. For example, the paddle 120 may experience resistance in mixing the bone cement components in the mixing chamber 118 and the resistance of the paddle 120 may be transferred through the gear assembly 136 to the rotor 116. The controller 144 may compensation by alternating the power being supplied. The controller 144 may also be configured to terminate power supplied to the stator 110 based on one of the signals from the sensor 142 being a current spike indicative that a piston 134 is prevented from further translation within the mixing chamber 118, and thus, the transfer of bone cement to the reservoir 132 of the delivery device 130 is complete.

As appreciated, the mixing of the bone cement components and transfer of the mixed bone cement to the delivery device 130 may be accomplished while the mixing chamber 118, the paddle 120, and the delivery device 130 are sealed within the shell 114 and without subsequent interaction by the user. The user may unseal the shell 114 by removing the cover 124 from the shell base 122 to access the delivery device 130 that has the bone cement stored in the reservoir 132. A sterile user, for example a scrub nurse in the sterile field, may retrieve the delivery device 130 from the shell 114 for use in the surgical procedure. The sterile transfer was facilitated in a relatively seamless workflow. After use, the disposable assembly 104, whereas the capital assembly 102 may be reused for subsequent procedures with a different disposable assembly 104.

A second implementation of the disposable assembly 304 is shown in FIGS. 6-11, with like components indicated by like numerals, plus one hundred (100), in which a driven shaft 316 is rotated by mechanical power transfer rather than inductive power transfer. The mechanical power transfer is accomplished across the sterile barrier. Referring first to FIGS. 6 and 7, the capital assembly 302 includes the base housing 306 and a motor (not shown) coupled to the base housing 306. The capital assembly 302 also includes a rotatable drive shaft 310 extending from the base housing 306, and a base coupler 340 The base coupler 340 is configured to removably engage the shell coupler 338 of the shell 314 with the rotatable drive shaft 310 engaging the driven shaft 316. The shaft-to-shaft connection between the rotatable drive shaft 310 and the driven shaft 316 permits a gear assembly, if provided, to be disposed in the capital assembly 302 instead of the disposable assembly 304.

The disposable assembly 304 includes the shell 314, and the mixing chamber 318 disposed within the shell 314. The cover 324 is removably coupled to the base 322 of the shell 314 to enclose the mixing chamber 318 within the shell 314. The shell 314 may define a recess 311 for receiving the drive shaft 310 of the capital assembly 302 and a portion of the base housing 306. The recess 311 is shown on a bottom of the shell base 322. The position of the recess 311 on the bottom of the shell 314 permits the shell 314 to be coupled to the base housing 306 while also being supported by the same surface that is supporting the capital assembly 302. It is also contemplated that the recess 311 may be positioned along a side of the shell base 322. In an alternative variant, it is contemplated that the capital assembly 302 may be handheld.

A removable shroud 313 may be removably coupled to the base 322 to cover the recess 311. The shroud 313 may be coupled to the base 322 via adhesive or other suitable means. The shroud 313 may be, for example, Tyvek® configured to be peeled away from the base 322. The driven shaft 316 and the shell coupler 338 may be at least partially disposed in the recess 311. As such, the shroud 313 may be configured to prevent access to the driven shaft 316 and the shell coupler 338 until indicated to do so. Therefore, a chamber defined within the recess 311 may be sterile until the shroud 313 is removed. In other words, the use of the shroud 313 in addition to the cover 324 provides access to two different areas of the shell 314 with a continuous sterile barrier therebetween.

The mixing chamber 318 may also be disposed adjacent the recess 311 opposite a portion of the shell 114 such that the driven shaft 316, and a center of the mixing chamber 118 is coaxial. In some configurations, the walls of the shell 314 that define the recess 311 may include the shell coupler 338. The driven shaft 316 is rotatably coupled to the shell 314 and configured to receive torque from the drive shaft 310 of the capital assembly 302 when the shell 314 is coupled to the base housing 306. In one implementation, the rotatable drive shaft 310 and the driven shaft 316 may collectively form a splined connection to prevent relative rotation between the shafts. The splined connection may permit translation of the driven shaft 316 to rotate and/or translate the paddle in the mixing chamber 318. In other configurations, the rotatable drive shaft 310 and the driven shaft 316 may include other forms of coupling sufficient to transfer torque from the rotatable drive shaft 310 to the driven shaft 316.

As discussed above, the bone cement components may be preloaded and/or loaded into the mixing chamber 118. In one example, the disposable assembly 304 may include an injection chamber 346 in selective communication with the mixing chamber 318 (see FIG. 7). The mixing chamber 318 being preloaded with one of the bone cement components (e.g., the powdered polymer), and the injection chamber 346 may be preloaded with the other bone cement component (e.g., the liquid monomer). A monomer injection apparatus, similar to those previously described, may be configured to cause the liquid monomer to be moved from the injection chamber 346 and into the mixing chamber 318. In this instance, the monomer injection apparatus may be disposed on the capital assembly 302. In other words, the capital assembly 302 need not be limited to mixing and transferring the bone cement, but also for combining the bone cement components. For example, the recess 311 of the shell 314 may include a second shaft interface, and the capital assembly 302 may include a second drive shaft functioning as the monomer injection apparatus. With the coupling of the disposable assembly 304 to the capital assembly 302, the second drive shaft removably engages the second shaft interface (in addition to the drive shaft 310 engaging the driven shaft 316). Suitable gearing within the capital assembly 302 may be activated in sequence to (i) operate the monomer injection apparatus, (ii) operate the drive shaft 310 at high-speed, low-torque for mixing the bone cement components, and (iii) operate the drive shaft 310 at low-speed, high-torque for transferring the bone cement to the delivery device 330. In still another example, the drive shaft 310 may be a singular shaft configured to operate in different directions and/or at different speeds to facilitate the steps. The aformentioned modes for mixing the bone cement components may be performed upon actuation of the actuator 348, or automatically by the controller based on signals from a sensor (e.g., a hall effect sensor) indicative that the shell 314 is coupled to the base housing 306.

Other alternatives for combining the bone cement components are contemplated. For example, the monomer injection mechanism, such as a spring-loaded plunger may be disposed within the recess 311 and automatically actuated upon coupling the disposable assembly 304 to the capital assembly 302. Alternatively, the monomer injection mechanism may be a syringe-like mechanism disposed within the recess 311 and manually actuated prior to coupling the disposable assembly 304 to the capital assembly 302. In particular, once the shroud 313 is removed, the monomer injection mechanism is configured to receive an input from the user (e.g., twist input, push input, etc.) to cause the preloaded liquid monomer to be injected and directed into the mixing chamber 318. In still another example, the shell base 322 may define the port (not shown), such as a Luer fitting, configured to accept the liquid monomer.

Another exemplary method of sterile handling within the surgical suite is represented in FIGS. 6-11. The method may include the step of removing the shroud 313 from the disposable assembly 304, as shown in FIG. 6. Further, the method may include the step of combining the bone cement components according to one of the exemplary aspects disclosed herein, and coupling the disposable assembly 304 to the capital assembly 302, as shown in FIG. 7. One or more of the above steps may be performed in a non-sterile area.

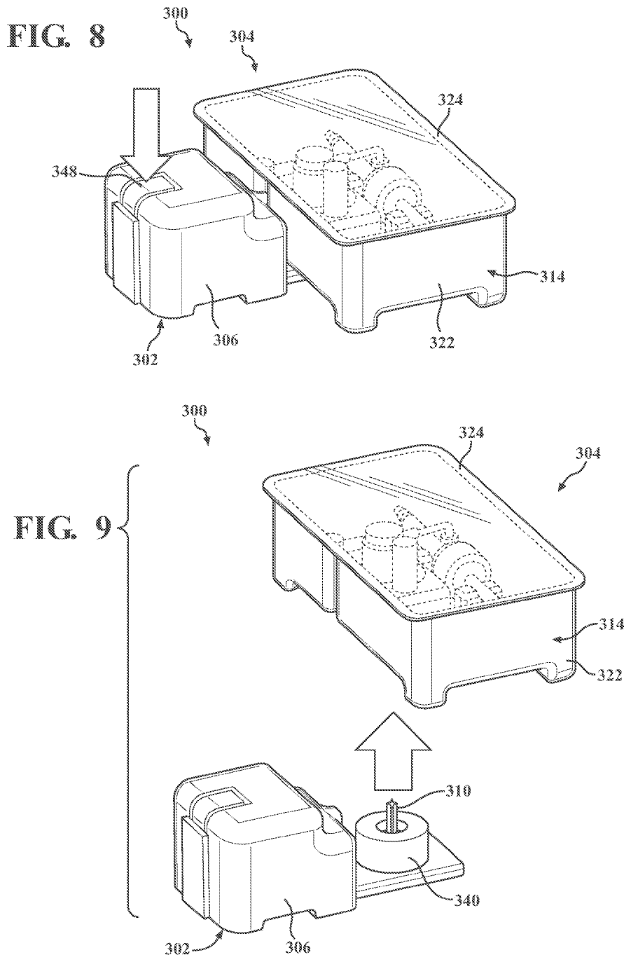

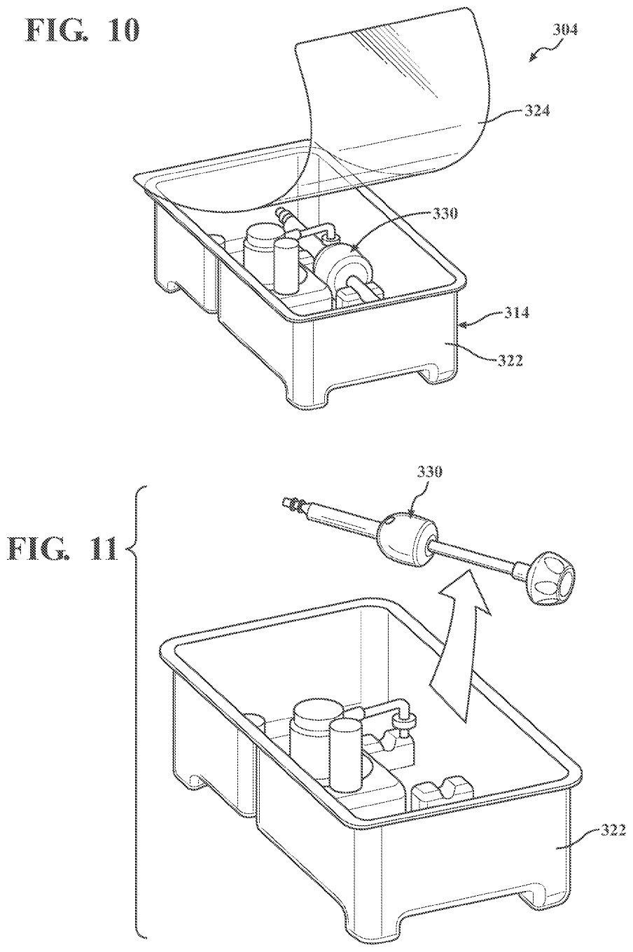

FIG. 8 shows the actuator 348 being engaged to initiate the operational cycle. The capital assembly 302 begins mixing of the bone cement components into and transferring the bone cement to the delivery device 330. Again, the mixing of the bone cement components and transferring of the mixed bone cement to the delivery device 330 is accomplished with the components sealed within the shell 314 and without subsequent interaction by the user The user may then decouple the disposable assembly 304 from the capital assembly 302, as shown in FIG. 9. Referring to FIG. 10, the user may unseal the shell 314 by removing the cover 324 from the base 322 to access the delivery device 330. A sterile user, for example a scrub nurse in the sterile field, may retrieve the delivery device 330 from the shell 314 for use in a surgical procedure, as shown in FIG. 11, in which case the sterile transfer is facilitated in a seamless and intuitive workflow.

A third implementation of the bone cement system 400 is shown in FIGS. 12-15 in which a handheld driver 402 functions as the capital assembly, and separately engages the mixing device and the delivery device 430 to perform the steps of mixing and delivery the bone cement, respectively. The driver 402 includes a motor, and a driver coupler 410 (e.g., a chuck) configured to transfer torque from the motor. The driver 402 is typically configured as a handheld driver to be grasped by a user during use. As such, the driver 402 may be disposed within a flexible aseptic enclosure 406 during operation so that the driver 402 may be reused for subsequent procedures. More specifically, a user may grasp and operate the driver 402 such that the flexible enclosure 406 is disposed between the user's hand and the driver 402 during operation.

The disposable assembly 404 includes the mixing chamber, and a mixing driven shaft 416a may be coupled to the paddle and the mixing chamber 418. The mixing driven shaft 416a includes a mixing coupler 426a at a proximal end of the mixing driven shaft 416a that is spaced from the mixing chamber 418. The mixing coupler 426 is configured to be coupled to and receive torque from the driver coupler 410 to rotate and translate a paddle within the mixing chamber 418. Mixing and transferring of bone cement components to the delivery device 430 may be accomplished in a similar manner as described above.

The delivery device 430 is removably coupled to the mixing chamber 418. The delivery device 430 has a delivery driven shaft 416b that is operable to force bone cement from the delivery device 430. The delivery driven shaft 416b includes a delivery coupler 426b configured to couple to and receive torque from the driver coupler 410 for introducing bone cement received from the mixing chamber 418 to a surgical site. The delivery and mixing couplers 426a, 426b are configured to separately attach to the same driver coupler 410 to permit a single driver 402 to perform both mixing bone cement in the mixing chamber 418 and application of bone cement from the delivery device 430. Using the driver coupler 410 to attach to the mixing coupler 426a and the delivery coupler 426b mitigates the number of actuators needed in the system 400 to reduce waste, cost, and complexity in having individual actuators dedicated to a single device.

As best shown in FIGS. 13 and 14, the disposable assembly 404 may include a bone cement component injector 446 coupled to the mixing chamber 418 to introduce a bone cement component to the mixing chamber in similar manners to the injection chamber 346 and the monomer injection mechanisms from the earlier described configurations. The bone cement component injector 446 includes an actuator 448 operable to move the bone cement component into the mixing chamber 418. The bone cement injector 446 may be detachable from the mixing chamber 418. Specifically, the bone cement injector 446 may be preloaded with the liquid monomer, and the mixing chamber may be preloaded with the powdered polymer, or vice versa. The actuator 448 may include a syringe configuration having a plunger 450 and a barrel 452. The user may depress the plunger 450 to decrease the volume in the bore of the barrel 452 to move the bone cement component into the mixing chamber 418.

An exemplary method of using the bone cement system 400 of FIGS. 12-15 includes the user unsealing a cover 424 from a shell 414 to access the mixing chamber 418, injector 446, and delivery device 430, as shown in FIG. 12. The user may remove the device, and couple the driver coupler 410 to the mixing coupler 426a, as shown in FIG. 13. The user then depresses the plunger 450 to move the bone cement component from the barrel 452 into the mixing chamber, as shown in FIG. 14. The user may operate the driver 402 to mix the bone cement components into bone cement and transfer the bone cement to the delivery device 430. The user decouples the driver coupler 410 from the mixing coupler 426a, and couples the driver coupler 410 to the delivery coupler 426b. The delivery device 430 may be operated with the driver 402 to deliver the bone cement.

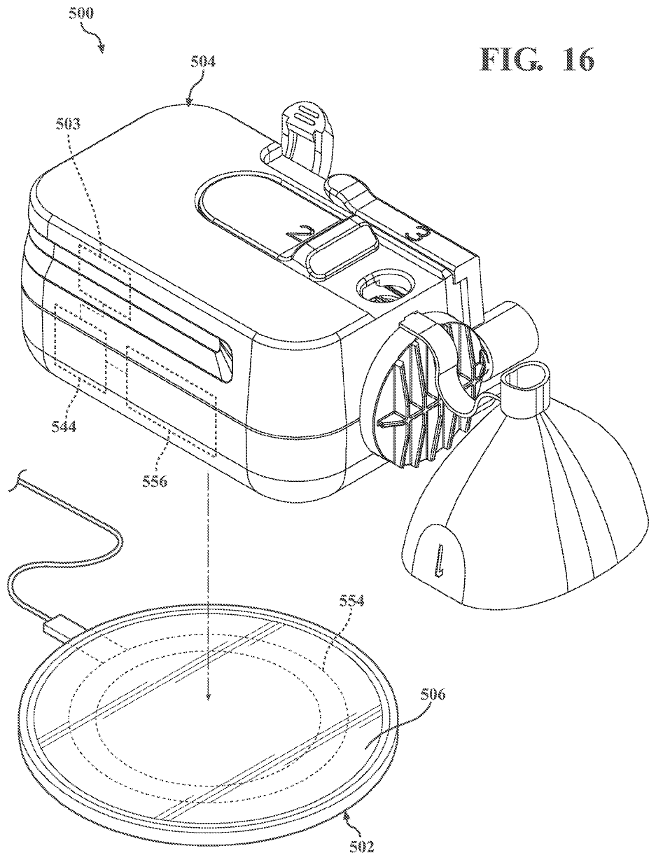

A fourth implementation of the bone cement system 500 is shown in FIG. 16. The disposable assembly 504 may be powered by inductive power coupling between the disposable assembly 504, and a power pad 502. The power pad 502 may include a pad body 506 for supporting the housing 514 of the disposable assembly. The power pad 506 may include a power transmitter 554 coupled to the pad body 506 for generating a magnetic field. The disposable assembly 504 may be similar to the assembly disclosed in the aformentioned International Patent Publication No. WO 2020/252296, in which the disposable assembly 504 may include a mixing chamber 518, a paddle rotatable within the mixing chamber 518, a motor 503 operably coupled to the paddle, and a controller 544 configured to operate the motor 503. In the present implementation, the disposable assembly 504 also includes a power receiver 556 coupled to the controller 544 and the motor 503 and configured to collectively form a power transformer with the power transmitter 554 when the housing 514 is supported by the pad body 506. While each of the power transmitter 554 and the power receiver 556 are shown schematically, it is appreciated that the transmitter 554 and receiver 556 may include one or more coils to facilitate inductive power coupling between the power pad 506 and the disposable assembly 504. An exemplary manner by which the inductive power coupling may be facilitated is disclosed in United States Patent Publication No. 2009/0096413, published Apr. 16, 2009, the entire contents of which are hereby incorporated by reference.

The power receiver 556 may receive power inductively through the housing 514 from the power pad 506 and provide power to the motor 503 in the disposable assembly 504 to rotate the paddle within the mixing chamber 518. It is contemplated that the inductive power received through inductive coupling may be transferred to power the motor 503 instead of or in addition to a battery on the disposable assembly 504. The motor 503 may include a direct current (DC). The use of a DC motor may be simpler in construction and less expensive than alternative current (AC) motors, while also providing for the power pad 502 being battery-or wall-powered. One of the power pad 502 and the disposable assembly 504 may have a switch to selectively supply power to the motor 503 when the disposable assembly 504 is supported by the pad body 506.

While bone cement compositions have been described as including the liquid monomer component and the powdered polymer component, other exemplary bone cement components may be mixed in accordance with the methods and systems described above, including those that include more than two components, those that include two liquid components, or those that include one or more paste components. In addition, the systems and methods described above may be used to deliver compositions other than bone cement, such as bone graft material, biological agents, other hardenable substances, and combinations thereof.

Several configurations have been discussed in the foregoing description. However, the configurations discussed herein are not intended to be exhaustive or limit the invention to any particular form. The terminology which has been used is intended to be in the nature of words of description rather than of limitation. Certain inventive aspects of the present disclosure are made with reference to the following exemplary clauses:

Clause 1—A capital assembly for a bone cement system, the capital assembly comprising: a base housing; a stator coupled to the base housing and defining an opening for removably receiving a rotor, the stator configured to generate a magnetic field for transferring inductive power to the rotor; a sensor coupled to the base housing and configured to generate signals indicative of a current draw on the stator, wherein the current draw on the stator is based on resistance from bone cement components being mixed within a mixing chamber; and a controller coupled to the base housing and in communication with the sensor, wherein the controller is configured to adjust power supplied to the stator based on the signals.

Clause 2—The capital assembly of clause 1, wherein the controller is further configured to terminate the power supplied to the stator based on one of the signals from the sensor being a current spike indicative that a piston is prevented from further translation within the mixing chamber.

Clause 3—A bone cement system comprising: a capital assembly comprising a base coupler; a disposable assembly comprising: a shell; a shell coupler coupled to the shell and configured to engage the base coupler of the capital assembly; a mixing chamber disposed within the shell; a cover removably coupled to the shell to seal the mixing chamber within the shell; a driven shaft rotatably coupled to the shell; and a paddle rotatably coupled to the driven shaft and rotatably disposed within the mixing chamber, wherein the capital assembly is configured to rotate the driven shaft of the disposable assembly to form bone cement with the mixing chamber enclosed within the shell.

Clause 4—The bone cement system of clause 3, wherein the capital assembly further comprises a motor, and a rotatable drive shaft coupled to the motor and configured to engage the driven shaft.

Clause 5—The bone cement system of clause 4, wherein the shell defines a recess for receiving the rotatable drive shaft of the capital assembly, and wherein the shell coupler is disposed in the recess.

Clause 6—The bone cement system of clause 3, wherein the capital assembly further comprises a stator coupled to the base housing, and wherein the driven shaft of the disposable assembly is a rotor configured to operably engage the stator.

Clause 7—The bone cement system of clause 6, wherein the rotor is rotatable relative to the shell and the stator in response to inductive power being transferred through the shell from the stator.

Clause 8—The bone cement system of any one of clauses 3-7, wherein the disposable assembly further comprises an injection chamber coupled to the mixing chamber, the injection chamber being preloaded with a first of the bone cement component and the mixing chamber being preloaded with a second of the bone cement component.

Clause 9—The bone cement system of claim 8, wherein the disposable assembly further comprises a monomer injection mechanism configured to move the first bone cement component from the injection chamber to the mixing chamber.

Clause 10—The bone cement system of clause 9, wherein the capital assembly further includes: a sensor configured to generate signals indicative of a coupling between the shell coupler and the base coupler, and a controller coupled to the base housing and in communication with the sensor and the monomer injection mechanism, wherein the controller is configured to operate the monomer injection mechanism to move the first bone cement component from the injection chamber into the mixing chamber based on the signal from the sensor indicating the shell coupler is coupled to the base coupler.

Clause 11—A bone cement system comprising: a handheld driver having a motor, and a driver coupler coupled to the motor; a disposable assembly comprising: a mixing chamber; a paddle disposed within the mixing chamber; a mixing coupler coupled to the paddle and configured to be coupled to and receive torque from the driver coupler to rotate and translate the paddle within the mixing chamber; and a delivery device removably coupled to the mixing chamber, wherein the delivery device includes a delivery coupler configured to couple to and receive torque from the driver coupler for introducing bone cement received from the mixing chamber to a surgical site.

Clause 12—The bone cement system of clause 11, wherein the driver is configured to be disposed within a flexible enclosure.

Clause 13—A bone cement system comprising: a power pad comprising a transmitter; a bone cement mixing assembly comprising; a mixing chamber; a paddle rotatably disposed within the mixing chamber; a motor operably coupled to the paddle; a controller coupled to the motor and configured to operate the motor to cause rotation of the paddle; and a receiver coupled to the controller and the motor, wherein the transmitter and the receiver collectively form a transformer for inductive power coupling between the power pad and the bone cement mixing assembly.

Clause 14—The bone cement system of clause 13, wherein each of the transmitter and the receiver comprise a coil.

Clause 15—A method of forming bone cement with a bone cement system including a capital assembly and a disposable assembly, wherein the capital assembly includes a base housing, a stator, and a controller, and wherein the disposable assembly includes a shell within which a rotor, a mixing chamber, a piston, a paddle, and bone cement components are sealed, the method comprising: causing, with the controller, power to be supplied from a power source to the stator, wherein inductive power is transferred from the stator through the shell to the rotor; rotating the paddle coupled to the rotor to mix the bone cement components in the mixing chamber to form bone cement; translating the piston with the mixing chamber to compress the bone cement; detecting, with a sensor, a current spike indicative that the piston is prevented from further translation within the mixing chamber; and terminating, with the controller, the power supplied to the stator based on a signal from the sensor.

Clause 16—The method of clause 15, wherein the step of mixing the bone cement components is performed while an interior of the shell is sealed.

Clause 17—A method of forming bone cement with a bone cement system including a capital assembly and a disposable assembly, wherein the capital assembly includes a base housing, a motor, and a rotatable drive shaft, and wherein the disposable assembly includes a shell within which a mixing chamber, a paddle, a delivery device, and bone cement components are sealed with a shell cover, the method comprising: coupling the shell to the base housing; providing an input to an actuator to operate the motor to rotate the paddle within the mixing chamber to mix the bone cement components to form bone cement and transfer the bone cement to the delivery device while the shell is sealed; unsealing the shell to access the delivery device; and removing the delivery device from the shell.

Clause 18—The method of clause 17, further comprising the step of transferring the shell into a sterile field of an operating suite before the step of unsealing the shell to access the delivery.

Clause 19—The method of clause 17 or 18, wherein the step of unsealing the shell to access the delivery device occurs after the bone cement is mixed and transferred to the delivery device.

Clause 20—A method of forming bone cement with a bone mixing system including a driver and a disposable assembly including a mixing chamber having a paddle, and a delivery device, the method comprising: coupling a driver coupler of the driver to a mixing coupler of the mixing chamber; operating the driver to rotate the paddle within the mixing chamber to mix bone cement components to form bone cement and transfer the bone cement to the delivery device; decoupling the driver coupler from the mixing coupler; coupling the driver coupler to a delivery coupler of the delivery device; and operating the driver to introduce mixed bone cement to a surgical site.

Clause 21—The method of clause 20, wherein the disposable assembly further comprises a bone cement injector and further comprising the step of introducing a first bone cement component with the bone cement injector from outside the mixing chamber to a second bone cement component disposed inside the mixing chamber.

Clause 22—The method of clause 20 or 21, further comprising detaching the mixing chamber from the delivery device after the step of operating the driver to transfer bone cement to the delivery device.

1. A disposable assembly configured to be coupled to a capital assembly to form a bone cement system, the disposable assembly comprising:
a shell;
a mixing chamber disposed within the shell;
a rotor rotatably disposed within the shell and configured to be operably coupled with a stator of the capital assembly with the shell removably coupled to the capital assembly; and
a paddle coupled to the rotor and rotatably disposed within the mixing chamber,
wherein the rotor is configured to be rotated by inductive power being transferred from the stator to the rotor through a sterile barrier provided by the shell to rotate the paddle within the mixing chamber to form bone cement.

2. The disposable assembly of claim 1, further comprising a cover removably coupled to the shell, wherein the disposable assembly is operable to form the bone cement with the cover enclosing the mixing chamber within the shell.

3. The disposable assembly of claim 2, further comprising a delivery device disposed within the shell and removably coupled to the mixing chamber, wherein the disposable assembly is operable to transfer the bone cement to the delivery device with the cover enclosing the mixing chamber and the delivery device within the shell.

4. The disposable assembly of claim 1, further comprising a projection extending from the shell and defining a cavity sized to receive the rotor.

5. The disposable assembly of claim 4, further comprising a shell coupler configured to releasably secure the shell to the capital assembly.

6. The disposable assembly of claim 5, wherein the shell coupler is disposed on the projection.

7. The disposable assembly of claim 1, wherein the shell defines a recess, and wherein the shell coupler is disposed within the recess.

8. The disposable assembly of claim 4, further comprising a bearing disposed within the projection and supporting the rotor.

9. The disposable assembly of claim 8, wherein the shell is contoured to support the bearing.

10. The disposable assembly of claim 1, wherein the disposable assembly is devoid of a coupler, wherein magnetic forces from the rotor and the stator are configured to maintain coupling between the disposable assembly and the capital assembly.

11. (canceled)

12. A disposable assembly configured to be coupled to a capital assembly to form a bone cement system, the disposable assembly comprising:
a shell;
a mixing chamber disposed within the shell;
a cover removably coupled to the shell to enclose the mixing chamber within the shell;
a shell coupler coupled to the shell and configured to be removably coupled to a base coupler of the capital assembly;
a driven shaft rotatably coupled to the shell and configured to receive torque from a drive shaft of the capital assembly with the shell coupler coupled to the base coupler; and
a paddle disposed within the mixing chamber and coupled to the driven shaft,
wherein the paddle is configured to be rotatable within the mixing chamber to form bone cement with the cover enclosing the mixing chamber and the shell providing a sterile barrier.

13. The disposable assembly of claim 12, further comprising a delivery device disposed within the shell and removably coupled to the mixing chamber, wherein the paddle is configured to be rotatable within the mixing chamber to form bone cement with the cover enclosing the mixing chamber and the delivery device within the shell.

14. The disposable assembly of claim 13, wherein the shell defines a recess, and wherein the shell coupler is disposed within the recess.

15. The disposable assembly of claim 14, wherein the driven shaft is at least partially disposed in the recess.

16. The disposable assembly of claim 14, wherein the mixing chamber is disposed adjacent the recess opposite the shell so as to be coaxially aligned with the driven shaft.

17. The disposable assembly of claim 12, further comprising a shroud removably coupled to the shell and covering the recess.

18. A disposable assembly configured to be coupled to a capital assembly to form a bone cement system, the disposable assembly comprising:
a shell defining a recess;
a mixing chamber disposed within the shell;
a cover removably coupled to the shell to collectively provide a sterile barrier to the mixing chamber within the shell;
a shell coupler coupled to the shell and disposed within the recess, wherein the shell coupler is configured to be removably coupled to a base coupler of the capital assembly;
a driven shaft rotatably coupled to the shell and configured to be rotated by a drive shaft of the capital assembly; and
a shroud removably coupled to the shell to aseptically seal the recess.

19. The disposable assembly of claim 18, wherein the driven shaft is at least partially disposed in the recess.

20. The disposable assembly of claim 19, wherein the mixing chamber is disposed adjacent the recess opposite the shell so as to be coaxially aligned with the driven shaft.

21. The disposable assembly of claim 1, wherein the shell is a thermoformed blister pack.

22-25. (canceled)
